(19) United States
(12) Patent Application Publication
(10) Pub. No.: US 2005/0084486 A1
(43) Pub. Date: Apr. 21, 2005

(54) Composition comprising alkaline sphingomyelinase for use as a dietetic preparation, food supplement or pharmaceutical product

(75) Inventor: Claudio De Simone, Ardea RM (IT)

Correspondence Address: NIXON & VANDERHYE, PC, 1100 N GLEBE ROAD, 8TH FLOOR, ARLINGTON, VA, 22201-4714, US

(73) Assignee: VSL Phamaceuticals, Inc., Gaithersburg, MD (US)

(21) Appl. No.: 10/964,770

(22) Filed: Oct. 15, 2004

Related U.S. Application Data
(63) Continuation of application No. 09/960,652, filed on Sep. 24, 2001, now abandoned.
Application No. 09/960,652 is a continuation of application No. PCT/IT00/00230, filed on Jun. 7, 2000.

(30) Foreign Application Priority Data
Jun. 9, 1999 (IT) RM99A000376

(51) Int. Cl. A61K 38/47

(52) U.S. Cl. 424/94.61

(57) ABSTRACT

The invention relates to a composition which, depending on the user, may be taken as a nutritional, dietetic or strictly therapeutic preparation, comprising as its active substance alkaline sphingomyelinase which is capable of preventing or treating various pathological conditions including cancerous processes, inflammatory processes of the intestine, hypercholesterolaemia and infections with Helicobacter pylori.

The present invention relates to the use of alkaline sphingomyelinase for the preparation of compositions intended for nutritional, dietetic or strictly therapeutic use, together with the compositions made in this way.

Consequently, these compositions may be in the form of and may act as food supplements, dietetic bases or medicinal preparations proper, according to whether they are intended to act as bases or prophylactic treatments or as therapeutic preparations proper, depending on the particular individuals for whom the composition is intended.

Three different types of sphingomyelinase (SMase) have so far been identified.

There is an acidic sphingomyelinase, which is a lysosomal enzyme (with an optimum pH of 4.5-5), deficiency of which causes Niemann-Pick disease, and there is a neutral sphingomyelinase, with an optimum pH of 7.5, for which two iso-forms have been described. One of these iso-forms is located in the cytoplasmic membrane and depends on magnesium, while the other is contained in the cytosol and is independent of cations. Both the acidic and the neutral sphingomyelinase are found in many tissues and cells and are ubiquitous enzymes, regulating numerous cell functions.

The third type is called alkaline sphingomyelinase, because it is mainly active at pH 9. It is independent of magnesium and has been found both in intestinal brush borders and in the bile. Alkaline sphingomyelinase does not occur in the stomach, duodenum or pancreas but it is found in the intestine, especially in the distal part of the jejunum. A marked alkaline sphingomyelinase activity has also been observed in the colon and the rectum. High levels of alkaline sphingomyelinase are also found in the bile, but this seems to be peculiar to human beings. This twofold source of sphingomyelinase makes human beings very efficient in comparison with other creatures as regards the hydrolysis of sphingomyelin (SM) introduced via the diet. It has hitherto been thought that alkaline sphingomyelinase cannot be produced by intestinal bacteria, because no differences have been found between conventional and germ-free animals [see R. D. Duan, Scand. J. Gastroenterology, 33 (1998) pp. 673-683].

Apart from the alkaline sphingomyelinase that is present in the intestine and that present in the bile, no other alkaline sphingomyelinases are known that could be used to produce compositions intended for nutritional, dietetic or strictly therapeutic use. Moreover, acidic and neutral sphingomyelinase cannot be employed owing to their differing characteristics (see the following table).

TABLE

| | | Neutral SMase | Alkaline SMase |
|---|---|---|---|
| | Acidic SMase | cytoplasmic | human intestine |
| Location | lysosomes | membrane | and bile |
| Optimum pH | 5.5 | 7.4 | 9 |
| Mg⁺⁺-dependence | No | Yes | No |
| Trypsin | No | No | Yes |
| resistance | | | |
| Thermal | <40° C. | — | <50-60° C. |
| stability | | | |
| Substrate | endocytic SM | membrane SM | SM in food |

The use of sphingomyelinase for cosmetic and dermatological purposes is already known.

Japanese Patent No. 63 216,813 describes cosmetic compositions that contain sphingomyelinase and are intended for counteracting the physiological decrease of this enzyme that occurs in the skin on ageing, and for promoting its transformation into ceramide which, in turn, has a beneficial moisturizing effect on the epidermis.

International Patent Application PCT WO 98/22,082 describes the use of sphingomyelinase for the preparation of dermatological compositions suitable for treating skin disorders such as dermatitis, psoriasis, ichthyosis and similar conditions. Furthermore, this PCT application describes the preparation of sphingomyelinase from strains of Gram-negative bacteria, Gram-positive bacteria and lactic acid bacteria, with clear advantages over the previously known processes, which use the organs of higher animals, such as the brain and liver, as starting materials.

It has now been found, surprisingly, that some bacteria possess high levels of alkaline sphingomyelinase, and that their ingestion can be beneficial for the host. These bacteria can be ingested live or in the form of extracts, provided that these are enzymatically active, possibly in combination with other bacteria such as lactic acid bacteria, with SM and/or with foods containing SM.

One of the objects of the present invention is therefore to provide a dietetic, nutrient or pharmaceutical composition that comprises alkaline sphingomyelinase in an amount that is sufficient to exert a dietetic, nutritional or therapeutic effect in an individual who needs it.

In particular, this composition is suitable for the prevention and/or treatment of disorders connected with intestinal development, cancerous processes, disorders of the immune response, inflammatory and apoptosic processes of the intestine and its associated structures, disorders connected with cholesterol synthesis, disorders due to the hydrophobic nature of the surfaces of the gastrointestinal tract, allergic disorders of the gastro-intestinal tract, disorders relating to digestive processes, inflammatory intestinal diseases, polyposis, in particular familial polyposis, hypercholesterolaemia, infections with Helicobacter pylori, disorders of neonatal growth, disorders connected with intestinal homeostasis and diseases of the central and peripheral nervous systems.

The composition is also useful for use in pediatric diets and/or in enteral alimentation. In pediatric diets the composition may be administered, for example, in combination with artificial milk, condensed milk, soybean milk, powdered milk, partially umanized milk and baby foods in general.

The composition preferably contains alkaline sphingomyelinase of bacterial origin, and the bacteria containing the alkaline sphingomyelinase are chosen from amongst Gram-positive bacteria, Gram-negative bacteria and lactic acid bacteria, or from mixtures thereof.

More especially, the alkaline sphingomyelinase of the composition is obtained from lactic acid bacteria, and these are chosen from the group comprising Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus jensenii, Lactobacillus leichmannii, Lactobacillus minutus, Lactobacillus plantarum, Lactobacillus rogosae, Lactobacillus salivarius, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

The particularly preferred strain amongst these lactic acid bacteria is Lactobacillus brevis CD2, filed on Feb. 6, 1998 under access No. DSM 11,988 in the German Collection of Micro-organisms and Cell Cultures (DSM) in Braunschweig, Germany (“Deutsche Sammlung von Mikroorganismen und Zellkulturen GmbH”) under the Budapest Treaty, or mutants or derivatives thereof.

According to a preferred embodiment of the invention, the lactic acid bacteria are used in the composition as live, lyophilized or sonicated bacteria.

The composition preferably contains from 1×10² to 1×10¹³ CFUs of lactic acid bacteria per gram of composition.

A particularly preferred composition contains 200×10⁹ Streptococcus thermophilus, 150×10⁹ Bifidobacteria and 4×10⁹ Lactobacillus acidophilus per gram of composition.

The composition according to the invention can also contain bile acids, in particular ursodeoxycholic acid, pectin, sphingomyelin or its compounds, drugs or foods containing sphingomyelin, arginine deiminase, fatty acids, polyunsaturated fatty acids, non fermented sugars, in particular lactulose, cholesterol inhibitors, ceramidase inhibitors, protease inhibitors, immunomodulators, anti-carcinogenic agents, vitamins, growth factors, surfactants, cereals, fibre, emulsifiers, stabilizers, lipids, antioxidants, preservatives, free-radical neutralizers and/or vaso-protectors.

The composition of the invention can be administered orally as a food supplement or orally or parenterally as a drug.

The invention also relates to the use of alkaline sphingomyelinase for the preparation of a dietetic, nutrient or pharmaceutical composition suitable for the prevention and/or treatment of disorders connected with intestinal development, cancerous processes, disorders of the immune response, inflammatory and apoptosic processes of the intestine and its associated structures, disorders connected with cholesterol synthesis, disorders due to the hydrophobic nature of the surfaces of the gastrointestinal tract, allergic disorders of the gastro-intestinal tract, disorders relating to digestive processes, inflammatory intestinal diseases, polyposis, in particular familial polyposis, hypercholesterolaemia, infections with Helicobacter pylori, disorders of neonatal growth, disorders connected with intestinal homeostasis and diseases of the central and peripheral nervous systems.

This composition is also useful for use in pediatric diets and/or in enteral alimentation. In pediatric diets the composition may be administered, for example, in combination with artificial milk, condensed milk, soybean milk, powdered milk, partially umanized milk and baby foods in general.

The alkaline sphingomyelinase used is preferably of bacterial origin, and the bacteria containing it are chosen from amongst Gram-positive bacteria, Gram-negative bacteria and lactic acid bacteria, or from mixtures thereof.

More especially, the lactic acid bacteria used are chosen from the group comprising Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus jensenii, Lactobacillus leichmannii, Lactobacillus minutus, Lactobacillus plantarum, Lactobacillus rogosae, Lactobacillus salivarius, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

The particularly preferred strain amongst these lactic acid bacteria is Lactobacillus brevis CD2, filed on Feb. 6, 1998 under access No. DSM 11,988 in the German Collection of Micro-organisms and Cell Cultures (DSM) in Braunschweig, Germany (“Deutsche Sammlung von Mikroorganismen und Zellkulturen GmbH”) under the Budapest Treaty, or mutants or derivatives thereof.

According to a preferred embodiment of the invention, the lactic acid bacteria are used in the composition as live, lyophilized or sonicated bacteria.

The composition used preferably contains from 1×10² to 10×10¹³ CFUs of lactic acid bacteria per gram of composition.

A particularly preferred composition contains 200×10⁹ Streptococcus thermophilus, 150×10⁹ Bifidobacteria and 4×10⁹ Lactobacillus acidophilus per gram of composition.

The following experiments were carried out to confirm the presence and efficacy of alkaline sphingomyelinase in the bacteria according to the present invention. These experiments involved the detection of alkaline sphingomyelinase, the enzyme responsible for the formation of ceramide in human skin.

### Methods

Assay of Acidic, Neutral and Alkaline Sphingomyelinase in Lactic Acid Bacteria and in Intestinal Biopsy Material

10 mg of lyophilized Streptococcus thermophilus bacteria were suspended in 500 μl of a buffer containing 50 mM Tris-HCl, pH 7.4, 10 mM MgCl₂, 2 mM EDTA, 5 mM DTT, 0.1 mM Na₃VO₄, 0.1 mM Na₂MoO₄, 30 mM p-nitrophenyl phosphate, 10 mM β-glycerophosphate, 750 mM ATP, 1 μM PMSF, 10 μM leupeptin, 10 μM pepstatin (from Sigma Chemical Co.) and 0.2% Triton X-100 (to assay the activity of neutral SMase) or 500 μl of 0.2% Triton X-100 (to assay the activity of acidic SMase). To assay the alkaline SMase, the bacteria and the (homogenized) intestinal biopsy material were suspended in a 0.25 M sucrose buffer containing 5 mM MgCl₂, 0.15 M KCl, 50 mM KH₂PO₄, 1 mM PMSF and 1 mM benzamidine (pH 7.4). The samples prepared in this way were then subjected to lysis by sonication (for 30 min during which, 10-sec “on” periods alternated with 10-sec “off” periods), using a Vibracell sonicator (Sonic and Materials Inc., Danbury, Conn.). The sonicated samples were then centrifuged for 30 min at 14,000 rpm at 4° C., the supernatant was removed, and the protein concentration was determined with a kit made by Bio-Rad Laboratories, (Richmond, Calif.).

To determine the neutral SMase, 100 μg of the sample were incubated for 2 hours at 37° C. in a buffer (final volume: 50 μl) containing 50 mM Tris-HCl, 1 mM MgCl₂, pH 7.4, and 2.25 μl of [N-methyl-¹⁴C]-sphingomyelin (SM) (0.2 μCi/ml, specific activity: 56.6 mCi/mmol, Amersham).

To determine the activity of the acidic sphingomyelinase, 100 μg of the bacterial lysate were incubated for 2 hours at 37° C. in a buffer (final volume: 50 μl) containing 250 mM sodium acetate, 1 mM EDTA, pH 5.0, and 2.25 μl of [N-methyl-¹⁴C]-SM.

To assay the alkaline SMase, the samples were added to 375 μl of Tris-EDTA buffer (pH 9) to a final volume of 0.4 ml, containing 50 mM Tris, 0.15 M NaCl, 2 mM EDTA and a mixture of 3 mM bile salts with a TC:TDC:GC:GCDC molar ratio of 3:2:1.8:1. This mixture of bile salts had been found to possess the highest stimulatory effect on alkaline SMase. The addition of EDTA to the buffer served to inhibit the activity of neutral SMase, which is Mg⁺⁺-dependent with an optimum pH of 7.5. The ¹⁴C-SM was dissolved in ethanol, dried under nitrogen and suspended in the assay buffer, containing a mixture of 3% Triton X-100 and 3 mM bile salts.

The reaction was terminated by the addition of 2 ml of a 2:1 mixture of chloroform and methanol. The phospholipids were extracted and analysed on TLC plates, while the hydrolysis of the SM was quantified by autoradiography and liquid scintillation counting. The SMase present in the sonicated bacteria and in the intestinal biopsy material was expressed as pmol of SM hydrolysed per hour per milligram of protein.

Activity of SMase from Streptococcus thermophilus

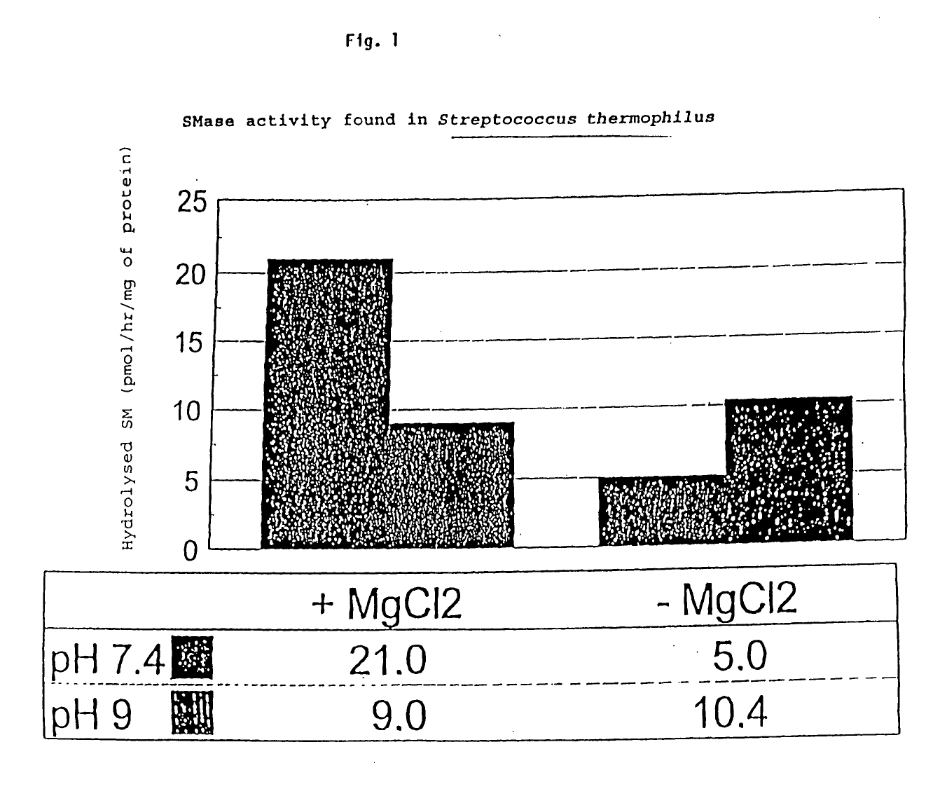

FIG. 1 shows the activity levels of sphingomyelinase in sonicated lactic acid bacteria. No activity due to acidic SMase was found, but appreciable levels of both neutral and alkaline SMase were observed in the bacterial samples tested under the experimental conditions used (various pH values and with and without MgCl₂).

Alkaline SMase Found in Intestinal Biopsy Material

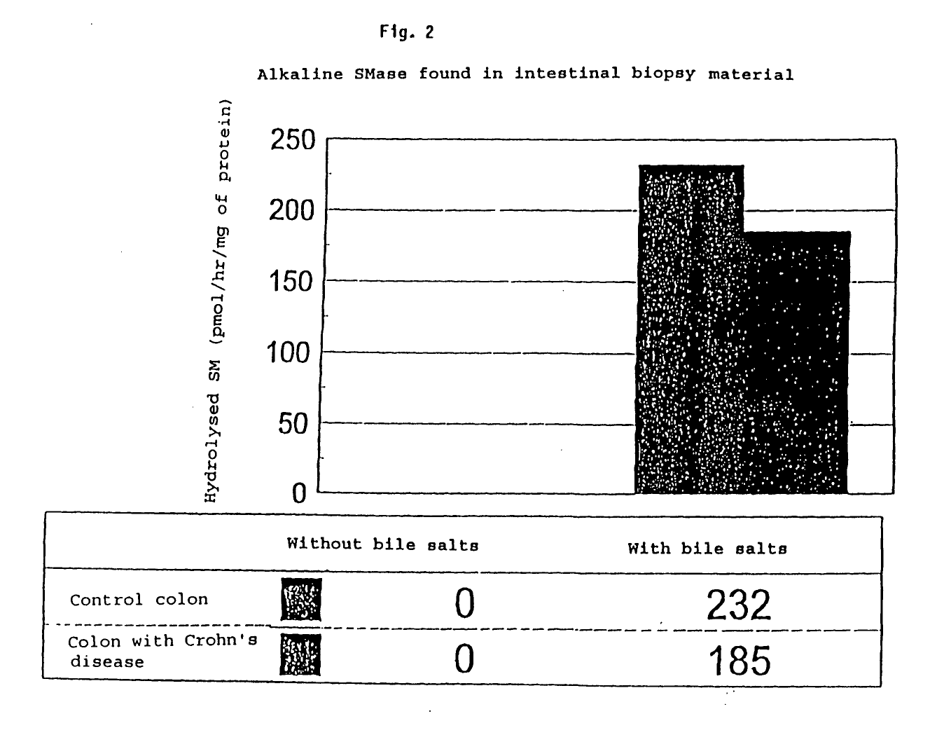

FIG. 2 shows that the analysis of SMase activity in the intestinal biopsy samples showed a high activity of alkaline SMase of the kind dependent on bile salts, which could not be detected in the absence of bile salts. The levels of enzymatic activity in the tissues of a patient suffering from Crohn's disease showed a lower level of alkaline SMase than the control sample.

Effect of Streptococcus thermophilus on Intestinal Alkaline SMase

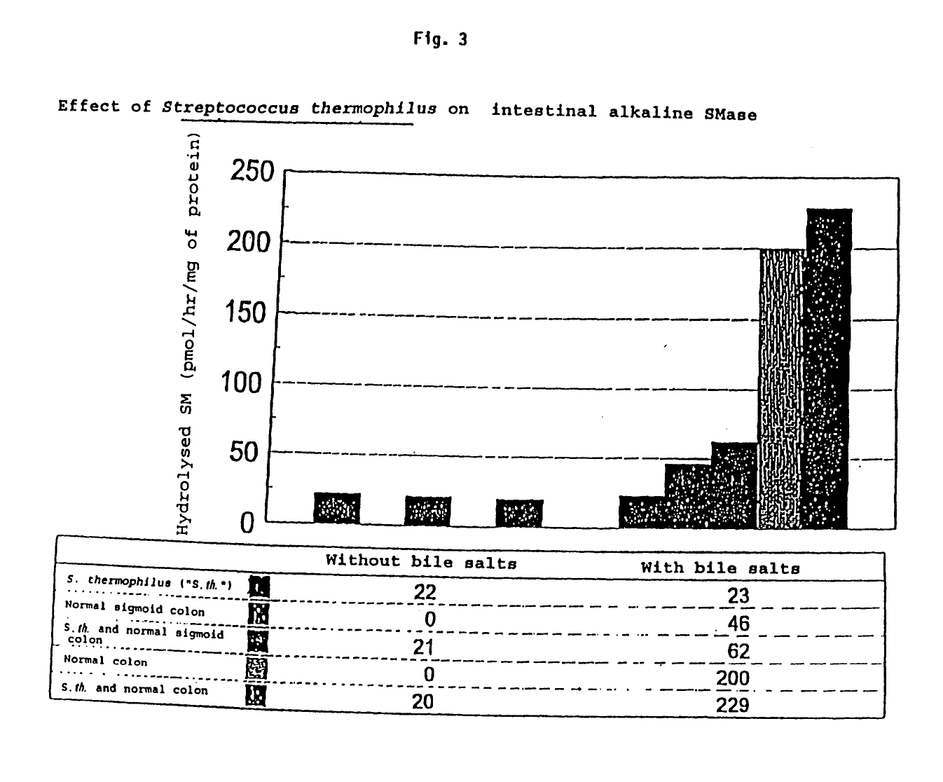

As shown in FIG. 3, the assay of the activity of SMase in the samples of Streptococcus thermophilus, under the experimental conditions used for the determination of intestinal SMase, showed that the bacterial enzyme was not affected by the presence or absence of bile salts. Furthermore, when the bacterial SMase activity and the intestinal SMase activity were tested simultaneously, the hydrolysis of SM increased additively. Similar results (not shown) were obtained with the Lactobacillus brevis CD2 strain, filed on Feb. 6, 1998 under access No. DSM 11,988 in the German Collection of Microorganisms and Cell Cultures in Braunschweig, Germany (“Deutsche Sammlung von Mikroorganismen und Zellkulturen GmbH”) under the Budapest Treaty, or mutants or derivatives thereof.

1. Dietetic, nutrient or pharmaceutical composition, characterized in that it comprises alkaline sphingomyelinase in an amount that is sufficient to exert a dietetic, nutritional or therapeutic effect in an individual who need it.

2. Composition according to claim 1, suitable for the prevention and/or treatment of disorders connected with intestinal development, cancerous processes, disorders of the immune response, inflammatory and apoptosic processes of the intestine and its associated structures, disorders connected with cholesterol synthesis, disorders due to the hydrophobic nature of the surfaces of the gastrointestinal tract, allergic disorders of the gastro-intestinal tract, disorders relating to digestive processes, inflammatory intestinal diseases, polyposis, in particular familial polyposis, hypercholesterolaemia, infections with Helicobacter pylori, disorders of neonatal growth, disorders connected with intestinal homeostasis and diseases of the central and peripheral nervous systems.

3. Composition according to claim 1, suitable for use in pediatric diets.

4. Composition according to claim 1, suitable for use in enteral alimentation.

5. Composition according to claim 3, characterized in that the composition is administered, in combination with artificial milk, condensed milk, soybean milk, powdered milk, partially umanized milk and baby foods in general.

6. Composition according to claim 1, characterized in that the alkaline sphingomyelinase is of bacterial origin, and the bacteria containing the alkaline sphingomyelinase are chosen from amongst Gram-positive bacteria, Gram-negative bacteria and lactic acid bacteria, or from mixtures thereof.

7. Composition according to claim 6, characterized in that the lactic acid bacteria are chosen from the group comprising Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus jensenii, Lactobacillus leichmannii, Lactobacillus minutus, Lactobacillus plantarum, Lactobacillus rogosae, Lactobacillus salivarius, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

8. Composition according to claim 7, characterized in that the Lactobacillus brevis CD2 strain is used, which is filed on Feb. 6, 1998 under access No. DSM 11,988 in the German Collection of Microorganisms and Cell Cultures (DSM) in Braunschweig, Germany (“Deutsche Sammlung von Mikroorganismen und Zellkulturen GmbH”), or mutants or derivatives thereof under the Budapest Treaty.

9. Composition according to claim 1, characterized in that the lactic acid bacteria are used in the composition as live, lyophilized or sonicated bacteria.

10. Composition according to claim 1, characterized in that it contains from 1×10² to 1×10¹³ CFUs of lactic acid bacteria per gram of composition.

11. Composition according to claim 7, characterized in that it contains 200×10⁹ Streptococcus thermophilus, 150×10⁹ Bifidobacteria and 4×10⁹ Lactobacillus acidophilus per gram of composition.

12. Composition according to claim 1, characterized in that it also contains bile acids, in particular ursodeoxycholic acid, pectin, sphingomyelin or its compounds, drugs or foods containing sphingomyelin, arginine deiminase, fatty acids, polyunsaturated fatty acids, non fermented sugars, in particular lactulose, cholesterol inhibitors, ceramidase inhibitors, protease inhibitors, immunomodulators, anti-carcinogenic agents, vitamins, growth factors, surfactants, cereals, fibre, emulsifiers, stabilizers, lipids, antioxidants, preservatives, free-radical neutralizers and/or vaso-protectors.

13. Composition according to claim 1, which can be administered orally as a food supplement.

14. Composition according to claim 1, which can be administered orally or parenterally as a drug.

15. Use of alkaline sphingomyelinase for the preparation of a dietetic, nutrient or pharmaceutical composition suitable for the prevention and/or treatment of disorders connected with intestinal development, cancerous processes, disorders of the immune response, inflammatory and apoptosic processes of the intestine and its associated structures, disorders connected with cholesterol synthesis, disorders due to the hydrophobic nature of the surfaces of the gastrointestinal tract, allergic disorders of the gastrointestinal tract, disorders relating to digestive processes, inflammatory intestinal diseases, polyposis, in particular familial polyposis, hypercholesterolaemia, infections with Helicobacter pylori, disorders of neonatal growth, disorders connected with intestinal homeostasis and diseases of the central and peripheral nervous systems.

16. Use according to claim 15 in pediatric diets.

17. Use according to claim 15, in enteral alimentation.

18. Use according to claim 16, characterized in that the composition is administered, in combination with artificial milk, condensed milk, soybean milk, powdered milk, partially umanized milk and baby foods in general.

19. Use according to claim 15, in which the alkaline sphingomyelinase is of bacterial origin, and the bacteria containing it are chosen from amongst Gram-positive bacteria, Gram-negative bacteria and lactic acid bacteria, or from mixtures thereof.

20. Use according to claim 19, characterized in that the lactic acid bacteria are chosen from the group comprising Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus jensenii, Lactobacillus leichmannii, Lactobacillus minutus, Lactobacillus plantarum, Lactobacillus rogosae, Lactobacillus salivarius, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

21. Use according to claim 20, characterized in that the Lactobacillus brevis CD2 strain is used, which is filed on Feb. 6, 1998 under access No. DSM 11,988 in the German Collection of Microorganisms and Cell Cultures (DSM) in Braunschweig, Germany (“Deutsche Sammlung von Mikroorganismen und Zellkulturen GmbH”) under the Budapest Treaty, or mutants or derivatives thereof.

22. Use according to claim 15, characterized in that the lactic acid bacteria are used in the composition as live, lyophilized or sonicated bacteria.

23. Use according to claim 15, characterized in that from 1×10² to 1×10¹³ CFUs of lactic acid bacteria are used per gram of composition.

24. Use according to claim 15, characterized in that 200×10⁹ Streptococcus thermophilus, 150×10⁹ Bifidobacteria and 4×10⁹ Lactobacillus acidophilus are used per gram of composition.
